(19) United States
(12) Patent Application Publication
(10) Pub. No.: US 2006/0064055 A1
(43) Pub. Date: Mar. 23, 2006

(54) Steerable devices

(76) Inventors: John Pile-Spellman, Pelham Manor, NY (US); Lei Feng, San Marino, CA (US)

Correspondence Address: BROWN, RAYSMAN, MILLSTEIN, FELDER & STEINER LLP, 900 THIRD AVENUE, NEW YORK, NY, 10022, US

(21) Appl. No.: 11/137,860

(22) Filed: May 24, 2005

Related U.S. Application Data
(60) Provisional application No. 60/573,861, filed on May 24, 2004.

(51) Int. Cl. A61M 31/00

(52) U.S. Cl. 604/95.05; 604/523

(57) ABSTRACT

The present invention provides steerable devices, such as catheters, having length, a proximal end, and a distal end that includes at least one shape memory structure incorporated into the device and configured such that graded or stepped bending of the shape memory structure may be achieved by variably heating the shape memory structure along some or all of the length of the shape memory structure to one or more transformation temperatures associated with the shape memory material either gradually or in steps, and driving devices providing the drive signal and/or energy to heat the shape memory structure to enable graded or stepped control thereof.

The present application claims priority to U.S. Provisional Patent Application No. 60/573,861, filed May 24, 2004, the disclosure of which is hereby incorporated herein.

### BACKGROUND OF THE INVENTION

The present invention relates to steerable devices, such as steerable catheters and/or probes that may be introduced into cavities and steered remotely to facilitate navigation through the cavities.

A number of minimally invasive procedures, such as angioplasty, stenting, thrombolysis, etc., involve accessing a site of interest via the subject's vasculature. Navigating catheters into blood vessel bifurcations may be difficult, however, particularly if the bifurcation requires an acute angle bend in the catheter for entry into the vessel. Although there are a variety of preshaped catheters available, with and without guidewires, such as L shaped, C shaped, the Simmons/Cobra shape, etc., a surgeon must select a preshaped catheter and guidewire according to the angle required to enter the bifurcation. This is problematic since the initial bend in the catheter may not be adequate for catheterization into vessels beyond the initial bifurcation, which may preclude navigation deeper into the distal vessel or result in damage to the distal vessel.

Existing steerable catheter/guidewires have numerous drawbacks. For instance, manually actuated steerable guidewires may be curved into a C shape by pulling an internal mandrel attached to the distal tip of the guidewire. Although this procedure may provide the necessary curve to enter vessels, the manually actuated guidewire is stiff and not easily torqued which consequently limits its overall usefulness. Electrically and mechanically actuated catheters are discussed in U.S. Pat. No. 5,090,956, entitled “Catheter with memory element-controlled steering,” U.S. Pat. No. 4,543,090, entitled “Steerable and aimable catheter,” U.S. Pat. No. 4,758,222, entitled “Steerable and aimable catheter,” U.S. Pat. No. 4,753,223, entitled “System for controlling shape and direction of a catheter, cannula, electrode, endoscope or similar article,” U.S. Pat. No. 6,447,478, entitled “Thin-film shape memory alloy actuators and processing methods,” U.S. Pat. No. 5,419,767, entitled “Methods and apparatus for advancing catheters through severely occluded body lumens,” and U.S. Publication No. 2002/0142119, entitled “Shape memory alloy/shape memory polymer tools,” each of which is hereby incorporated herein by reference in their entirety. However, these mechanically and electrically actuated catheters have limitations associated therewith, such as localized overheating due to the application of heat, for example with laser energy at a single location on a shape memory alloy actuator, abrupt bending, limited control with respect to the amount or location of the bending, complexity in production, etc., that similarly limit their usefulness. Accordingly, there is an ongoing need for steerable catheters/guidewires that overcome some or all of the limitations associated with existing steerable catheters/guidewires.

### SUMMARY OF THE INVENTION

The present invention provides steerable devices, such as catheters, having a length, proximal end, and a distal end that includes at least one structure composed of a material that exhibits shape memory characteristics, such as a shape memory alloy (“SMA”) or a shape memory polymer (“SMP”), which has the ability to return to a predetermined shape upon application of energy, such as heat. The structure, such as a wire, is generally incorporated into the device, such as within the distal end of a catheter, and includes a variable heating element or any other means for heating the structure, such as a heating coil or joule heating elements, configured such that graded bending of the structure in the direction of the predetermined shape may be achieved. Graded bending is generally achieved by variably heating the shape memory material or structure, along some or all of the length of the structure to one or more transformation temperatures associated with the shape memory material or materials, either gradually or in steps. The steerable device may include a plurality of shape memory structures with different predetermined shapes placed in different segments of the device to achieve complicated bends.

Variable heating and the consequential graded bending, for instance, may be achieved with a heating coil having a variable or essentially non-constant spacing or density, which may increase, linearly, non-linearly, in steps, or otherwise, in between the distal end and proximal end of the device or along at least a portion of the device. The heating coil density may decrease from the distal end to the proximal end, from the proximal to the distal end, or combinations thereof to provide actuation or movement beginning in the respective end or ends with the larger coil density. In one embodiment, the heating coil includes at least two coil densities to provide movement in at least two stages. Similarly, the heating coil may include at least three coil densities to provide movement in at least two stages. A thermal insulation coating may also be applied to the external side of the heating coil to allow for more efficient heating of the shape memory material.

Alternatively or in addition, variable heating may be achieved with a joule heating element, which, for example, may be the shape memory structure, where electrical current is passed through the joule heating element, such as an SMA wire, that has been configured to be heated up to one or more transformation temperatures gradually or in steps. In this instance, variable heating may be achieved by passing current through the joule heating element, such as the shape memory structure, having varied or essentially non- constant electrical resistance, such as due to a varying cross sectional areas or varying compositions, e.g., resistivity, in a graded fashion along the length of the shape memory structure, such as along the SMA wire. Accordingly, graded bending may be achieved by appropriately configuring the cross sectional area or composition along the length of the shape memory structure to provide the desired graded bending, e.g., proximal-to-distal, distal-to-proximal, combinations thereof, or otherwise.

The present invention further provides methods of navigating or orienting the steerable device, such as a catheter, through or into cavities, such as a subject's vasculature. This aspect of the invention is generally achieved by controlling the bend or bending of the steerable device in a graded manner such that the steerable device may be finely controlled to orient the device in one or more directions and degrees of angulations that facilitates, for example, navigation around sharp turns in a vessel. Graded bending of the steerable device may be achieved by variably heating a shape memory structure incorporated into the device so that the shape memory structure may achieve the desired bending for entry into the cavity or around a bend in the cavity. Once the desired entry is achieved, the bend may be relaxed to allow the steerable device to be navigated further into the cavity. The bend may be relaxed by either removing the heat supplied to the shape memory structure to allow the structure to cool and resume a relaxed shape, by applying heat to the remaining wires while the previously heated wire cools, or actively removing heat from the previously heated wire.

The present invention thus improves the versatility of, for example, steerable catheters by facilitating catheterization of vessels with different angulations. The gradual bending of the catheter can also improve the safety of steerable catheters by reducing the chance of large abrupt bending that may dissect the blood vessel. The present invention also provides a catheter with segmental design that can form a complex shape in vivo in a controlled manner, which facilitates catheterization of complex vascular trees, particularly branches that comes off at an acute or reverse angle. Additional aspects of the present invention will be apparent in view of the description that follows.

### BRIEF DESCRIPTION OF THE FIGURES

FIGS. 1a-1d depict a steerable device according to one embodiment of the invention;

FIGS. 2a-2d depict a steerable device according to another embodiment of the invention;

FIG. 3a-3c depict a steerable device according to another embodiment of the invention;

FIG. 4a-4c depicts a steerable device according to another embodiment of the invention; and

FIG. 5 depicts a catheter hub according to one embodiment of the invention.

### DETAILED DESCRIPTION OF THE INVENTION

The present invention provides steerable devices, which may be slender devices, with graded or gradual control capability. Generally, the steerable devices have a proximal end and a distal end that includes at least one structure composed of a material that exhibits shape memory characteristics, such as a shape memory alloy (“SMA”) or a shape memory polymer (“SMP”), which has the ability to return to a predetermined shape upon application of energy, such as heat, and a variable heating element or any other means for variably heating the structure. The shape memory structure is generally incorporated into the device and is configured so that graded bending of the structure may be achieved by variably heating the shape memory material, along some or all of the length of the structure, either gradually or in steps, to one or more transformation temperatures associated with the shape memory material or materials.

Although the present invention may be described by way of example in relation to catheters and in minimally invasive procedures, it is understood to those skilled in the art that the present invention is applicable to other devices, such as probes, endoscopes, robotic arms, etc., and applicable to procedures in non-medical fields, and is thus not limited thereto. Moreover, although the steerable device may be explained in connection with shape memory alloys, it is further understood that the invention may be practiced with various types of shape memory materials.

The present invention takes advantages of the physical properties of shape memory materials to make steerable devices, such as catheters, that can be controlled by variably heating the shape memory material, such as with electric current, to provide graded or gradual bending. In one embodiment of the present invention, a steerable device, such as a catheter, is provided including one or more shape memory structures, such as SMA wires, incorporated into, e.g., connected to or embedded in, at least a portion of the wall or walls along the length of the of the device, such as at the distal end of the steerable device.

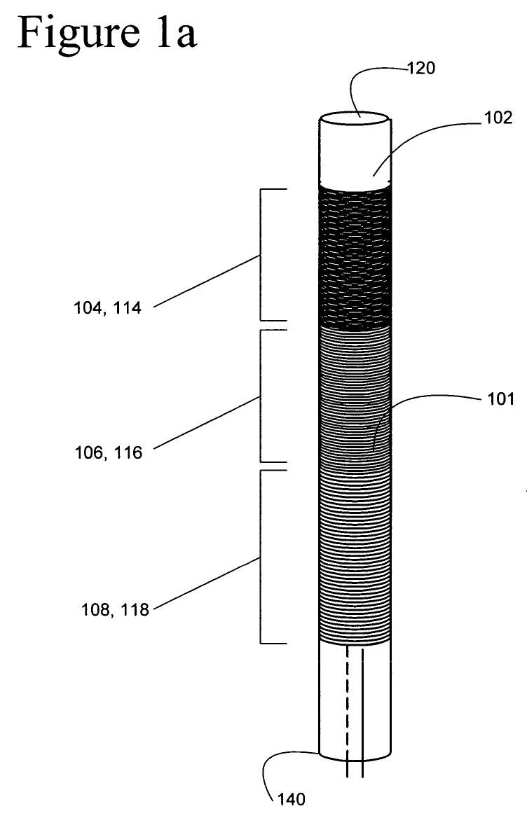

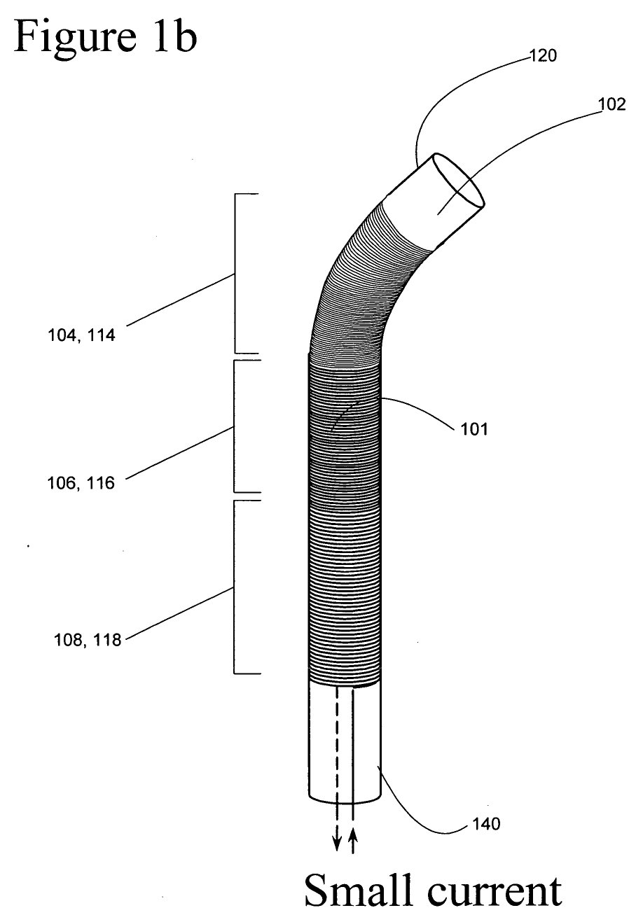

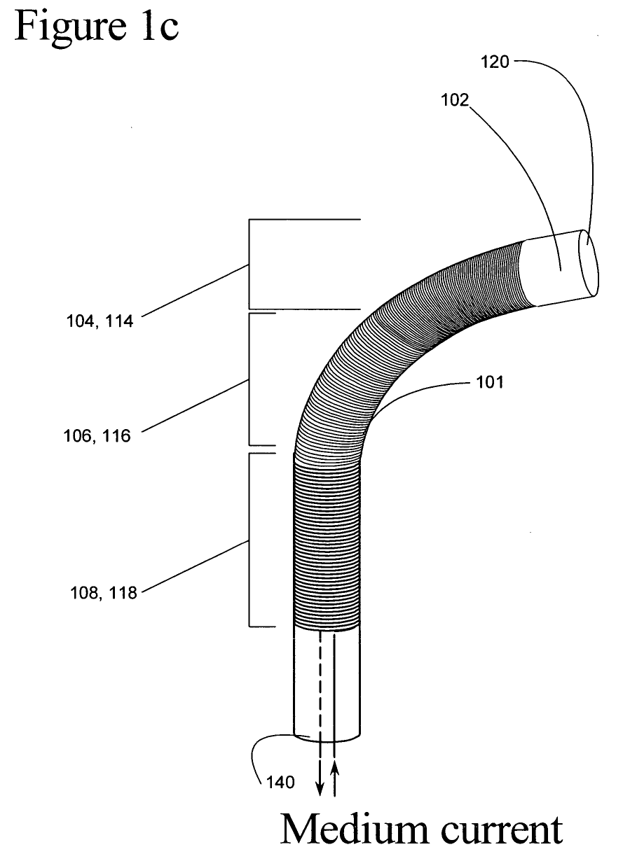

Referring to FIG. 1a, variable heating, for instance, may be achieved with a heating element, such as a heating coil 101, having a varied or essentially non-constant coil spacing or density configuration. For instance, the spacing between the coils, which may be wrapped about or near the shape memory structure, such as an SMA wire 102, or the steerable device, and may increase linearly, non-linearly, in steps, or otherwise, in between the distal end 120 and proximal end 140 of the steerable device. In one embodiment, the density of heating coil decreases gradually or in steps from the distal end 120 to the proximal end 140, e.g., from the tip to the shaft of a catheter, to provide actuation or movement beginning with the distal end 120 and continuing to the proximal end 140. In another embodiment, the heating coil includes at least two coil densities to provide movement in at least two stages. For example, the device may include three coil densities, 104, 106, and 108, wrapped about or within the SMA wire 102 or steerable device to provide movement beginning with a first stage 114, and continuing to a second stage 116 and third stage 118, as shown in FIGS. 1b-1d. The density of the heating coil may also be configured such that the density increases from the distal end 120 to the proximal end 140, e.g., from the tip to the shaft of a catheter, to allow the device to bend in a proximal-to-distal fashion. This design is particularly suitable for large (above 5 F) guiding catheters, which have relatively stiff walls to provide support to the micro-catheters. The heating coil 101 is generally electrically connected, for example, through copper or gold wires, to a driving device that generates an electric current to provide the variable movement as described herein. A thermal insulation coating may also be applied to the external side of the heating coil 101 to allow for more efficient heating of the shape memory material.

SMA, for instance, exists in either a low-temperature martensite phase or a high-temperature austenite phase. In the martensite phase, SMA has a comparatively low yield strength and thus is relatively flexible and ductile. When SMA is heated to its particular transformation temperature, SMA undergoes a change of crystal structure that causes the material to recover strain and thus to resume a predetermined shape, and also to gain yield strength and stiffness. The transformation of SMA from the martensite to the austenite phases can therefore generate force to actuate devices or instruments, such as by incorporating SMA material into the distal tip of catheters to bend the catheter tip into a predetermined shape or angle.

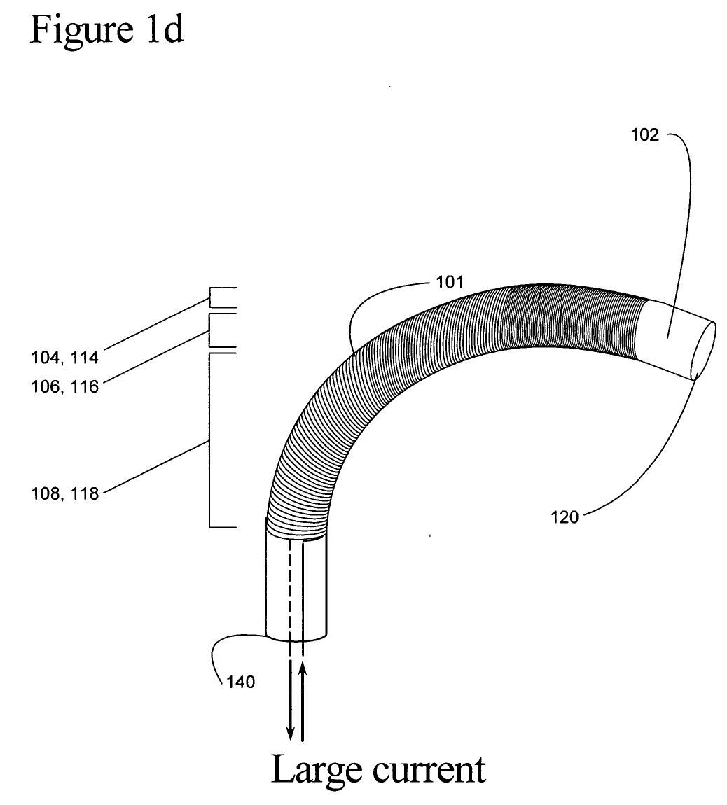

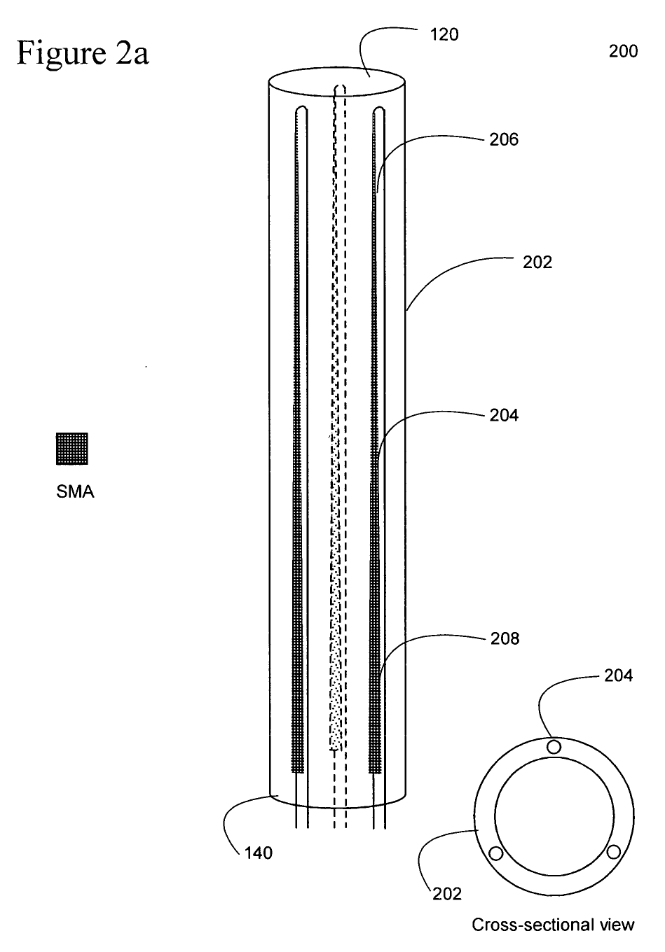

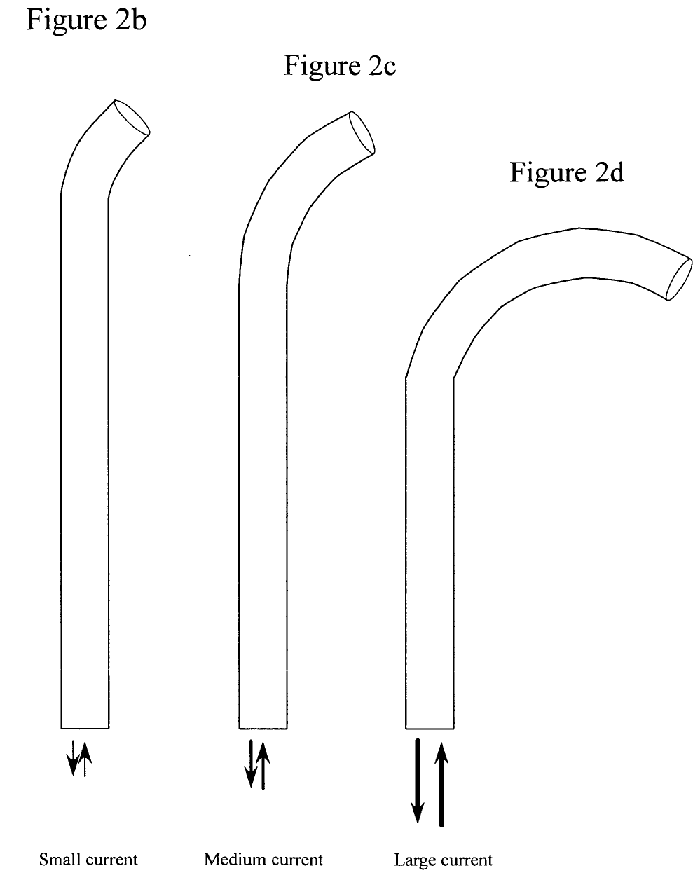

A variety of shape memory materials exhibiting shape memory properties may be used in connection with the present invention, including SMAs, such as nickel-titanium alloy (Nitinol). Nitinol, for instance, has higher electrical resistance than metals commonly used in electric circuits as conductive wires, such as copper and gold, yet has a small enough heat capacity, allowing it to be heated by electrical current. The heat conductivity is smaller than copper and gold, making it possible to generate a small temperature gradient in the alloy. Referring to FIG. 2a, for example, when current is passed through tapered diameter SMA wire 204, e.g., a wire having a first cross sectional area and at least one other cross sectional area different than the first, the end of smaller diameter or cross sectional area will heat up faster, due to higher resistance (proportional to the square of diameter) and lower mass per unit length (also proportional to the square of diameter). As current increases, the end with the smaller cross sectional area will reach the transformation temperature first and change to its predetermined shape before the end of the larger diameter.

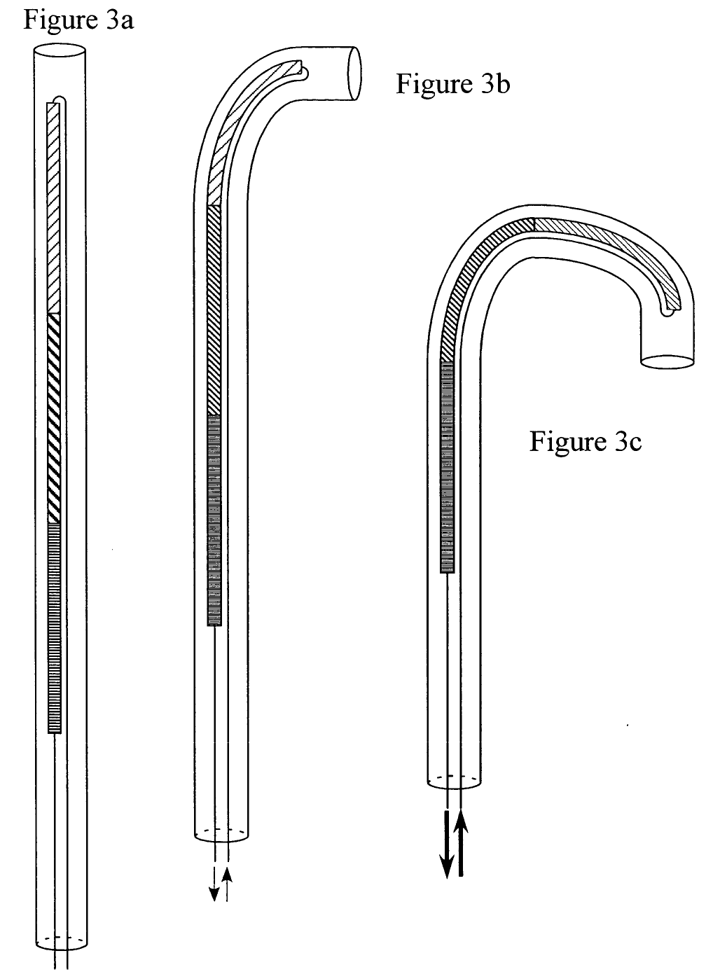

The transformation temperature and the electrical resistance can further be controlled to provide graded bending by altering the composition of the alloy. Referring to FIGS. 3a-3c, for instance, two or more SMA wires 302, 304, 306, having different compositions, e.g., resistivity, and requiring different levels of current to achieve the transformation temperature or having different transformation temperatures can thus be placed in tandem or in series in an electric circuit. Therefore, the shape change of these two or more segments of SMA wire can be controlled by the amount of current passed through the circuit.

The steerable device may generally be operatively connected to a driving device that provides the signal and/or energy, e.g., the current, to heat the shape memory material as described herein. In one embodiment, electrical current is applied to variably heat at least one of the SMA wires, causing the SMA wire to heat and bend in a desired direction and a desired degree toward the preformed shape of the SMA wire or wires, while the remaining wires remain in a cool relatively flexible or ductile stage.

In a three SMA wire embodiment, as shown in FIGS. 2a-2d, bending may thus be achieved by heating one of the SMA wires 204 while the remaining two wires remain in a cool state. A vector force is generated, with the application of heat to the SMA wire 204, in the direction of the preformed shape of the SMA wire, i.e., away from the surface of the device and perpendicular to the plane defined by the other two wires, and having a strength that is proportional to the energy input. A second SMA wire may similarly be heated to generate a second vector force. The overall force generated on the device by heating the two SMA wires is the summation of the two vector forces. Therefore, by selectively heating one or two of the three SMA wires to a variable degree, a vector force in any direction of the 360° space can be generated, to bend the device in a desired direction. The degree of bending, which is proportional to the strength of the vector force, can also be altered by proportionally changing the energy input to the heated SMA wires, in order to maintain the desired direction.

The steerable device, such as a catheter, may thus be controlled gradually or in a graded manner to navigate around sharp turns in a vessel. Navigation around a bend in a blood vessel is generally accomplished by gradually bending the device sufficient to engage the tip of the device with a selected blood vessel branch orifice. Once engaged, heating is stopped and the SMA wire or wires are allowed to cool thereby removing the vector force or forces that acted to bend the catheter are. As the SMA wire or wires relax, the catheter resumes its original shape and can be advanced further into the branch of the selected vessel. The catheter may also be returned to the original shape, e.g., essentially straight, quickly by applying heat to the remaining wires to counterbalance the vector force that bends the catheter.

It is understood that the shape memory structure or wire may be made in various cross sectional shapes, such as circular, elliptical, square, rectangular, tubular, etc. In one embodiment, a SMA wire is incorporated into the walls of the device is a relatively thin film and/or a tube. The SMA wire generally has a predetermined shape or bend that is resumed at its transformation temperature. The SMA wire should generally generate enough force to bend the wall of the devices, such as catheters, to the desired angle to facilitate, for example, catheterization of tortuous vessels. The transformation temperature of the SMA material may be set at a few degrees above the temperature of the cavity into which the device may be introduced so that the material is normally in the martensite phase while in the cavity. Thus, where the device, such as a catheter, is designed to be introduced into a subject's vasculature or other cavities, the SMA material may have a transformation temperature a few degrees, e.g., about 2 to 10 degrees Celsius, above the subject's body temperature such that it remains ductile at the body temperature. This aspect allows a user to control bending with minimal energy needed to raise the temperature for SMA material to bend into a predetermined shape.

As noted above, the steerable device of the present invention may be controlled gradually, for instance, with graded bending of at least a portion of the device to facilitate navigation therewith. Referring back to FIG. 1a, to control the steerable device, a current may be applied to the heating coil 101 to bring the shape memory structure above the transformation temperature. To bend the distal end 120 of the device 100, a first amount of current, sufficient to heat the portion of the SMA wire 102, for instance, representing the first stage 114 having a first coil density 104 to the transformation temperature, is passed through the heating coil to allow the distal end 120 of the SMA wire 102 to bend in the first stage 114 as shown in FIG. 1b. Similarly, a second and third amount of current may be passed through the heating coil to heat the SMA wire 102 representing the second stage 116 and third stage 118 to the transformation temperature to bend in the second and third stages 116, 118, respectively, as shown in FIGS. 1c and 1d. In the instance the heating coil density is graded between the ends of the SMA wire 102, a variable amount of current may be passed through the heating coil 101 to heat the SMA material to its transformation temperature gradually along the length of the SMA wire thereby providing a greater degree and gradual bending along the length of the wire by increasing the current passed through the coil 101.

For catheterization, the steerable device may be introduced into a subject's vasculature in a relaxed or relatively flaccid shape. At a bifurcation, a user enters an appropriate vascular branch by actuating or bending at least the distal end of the device as described herein. Once the device sufficiently engages the desired branch of the vascular tree with the bent tip, the relaxed or flaccid shape may be resumed for continued catheterization. The relaxed shape is generally resumed by allowing the SMA material to return to its martensite phase. This may be accomplished by discontinuing the heat applied to the material, e.g., the current passed to the heating coil, so that the device may be allowed to cool down by convection with blood flow. The cooling process may be accelerated, for example, by passing cold saline through a passage in the device, such as through the lumen of the catheter.

As noted above, the graded bending may be achieved by heating at least one of the SMA wires incorporated into the device either gradually or in steps. It is understood that the gradual or stepped heating may be achieved in various ways. For instance, the SMA wire maybe heated with joule heating elements, e.g., by passing current directly through the shape memory structure, such as the SMA wire. In this instance, variable heating may be achieved by varying the electrical resistance of the joule heating element, e.g., the shape memory structure or SMA wire, and consequentially the amount of current necessary to heat the element to its transformation temperature.

In one embodiment, the joule heating element is the shape memory structure and the electrical resistance of the joule heating element is varied with the element having a cross sectional area that increases in a graded manner, e.g., linearly, non-linearly, stepped, or otherwise, along the length of the device or the SMA wire. The electrical resistance of a wire and the heat produced by passing current there through is inversely proportional to the cross sectional area of the wire. Thus, the amount of current necessary to heat sections of the SMA wire with a larger cross sectional area is greater than that required to heat sections of the wire with a smaller cross sectional area. Accordingly, graded bending may be achieved by appropriately configuring the cross sectional area along the length of the joule heating element or the SMA wire to provide the desired graded or stepped bending, e.g., proximal-to-distal, distal-to-proximal, or otherwise. A combination proximal-to-distal and distal-to-proximal bending may be achieved with wires oriented so that each provides graded bending in opposite directions.

Referring to FIG. 2a, in one embodiment, the device 200 includes at least one joule element that is an SMA wire 204 having a varied or essentially non-constant cross sectional area that increases linearly, non-linearly, in steps, or otherwise, between the distal and proximal ends 206, 208 of the steerable device 200. A plurality of SMA wires 204 may be incorporated into the walls of the device 200 to allow a user to selectively bend one or more SMA wires 204 for greater control. In one embodiment, three SMA wires having tapering diameters are incorporated into the tip of a device 200, such as a catheter. The SMA wires may be electrically connected to a driving device that generates the current necessary to heat the SMA wire or wires. The driving device generally allows a user to vary the amount of current passed through the SMA wire or wires so that graded bending may be achieved in a selected direction.

In the instance the cross sectional area of the SMA wire increases from the distal end 120 to the proximal end 140, as current is supplied to the SMA wire, the distal end of the SMA wire will reach the transformation temperature first and, as the current increases, followed by longer segments of the SMA wire, which provides graded bending of the SMA wire in accordance with the predetermined shape of the SMA wire as shown in FIGS. 2b-2d. Where a plurality of wires are incorporated into the device, a desired bend in a three-dimensional space may be obtained by passing appropriate current to one or more of the individual SMA wires or channels.

The current required to generate the desired bend in the SMA wire partially depends on the difference between transformation temperature and the cavity temperature, e.g., the subject's body temperature, the yield strength of the material comprising the steerable device, the cross sectional area and force generated by the transforming SMA wire, and the relative heat conductivity of the SMA wire and the steerable device. Where the device is adopted for catheterization, the user may observe the bending in the device using imaging means, such as x-rays or magnetic resonance imaging. A three-dimensional road map of the vascular tree, generated by rotational digital subtraction angiography, computer tomography or magnetic resonance imaging, may also be input to the electrical driving device which automatically selects the appropriate SMA wire and the degree of heating that is necessary to direct the catheter tip to a target vessel.

The electrical resistance of the SMA wire may also be varied by composing the wire of a plurality of SMA compositions or by connecting a plurality of SMA wires having different compositions in series, such that graded bending thereof may be achieved. Referring to FIGS. 3a-3c, the device may include at least one SMA wire having varying resistances along the length of the SMA wire due at least in part to different SMA compositions. The graded bending may similarly be achieved with an SMA wire composed of a plurality of SMA compositions with different transformation temperatures. Thus, sections of SMA wire require different amounts of current to heat the respective sections of the wire to the one or more transformation temperatures, allowing a user to achieve graded bending by controlling the current passed through the composite wire.

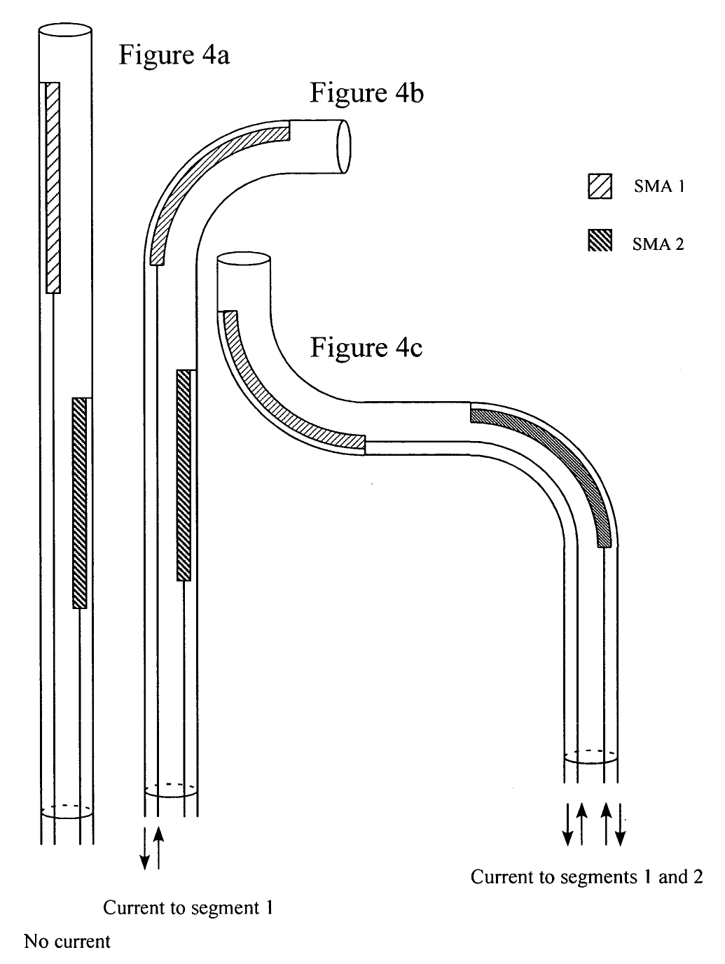

It is understood that a variety of bends and shapes may be achieved with the SMA wires by setting or training the wire to resume one of a variety of predetermined shapes, such as shapes in the form of C, S, L, shapes having acute, right, or obtuse angle bends, etc., or a combination thereof. In at least one embodiment of the present invention, the steerable device achieves one or more shapes with a plurality of SMA wires with different predetermined shapes placed in different segments of the device, as shown in FIGS. 4a-4c. The SMA wires may be electrically connected to the driving device so that each SMA wire may be heated individually or in combination to generate complicated shapes of the distal catheter at different times. The sequential generation of complicated shapes in the device, such as a catheter, can facilitate the catheterization of acute branches of the vascular tree. This design is particularly suitable for micro-catheters for the catheterization of intracranial vessels.

The steerable devices may be manufactured from a variety and/or a combination of biocompatible and non-biocompatible materials, such as polyester, Gortex, polytetrafluoroethyline (PTFE), polyethelene, polypropylene, polyurethane, silicon, steel, stainless steel, titanium, Nitinol, or other shape memory alloys, copper, silver, gold, platinum, Kevlar fiber, carbon fiber, etc. Where non-biocompatible materials may come into contact with the anatomic structure, the components made from the non-biocompatible materials may be covered or coated with a biocompatible material. In one embodiment, the device is made in part of a flexible biocompatible polymer, which allows a user to navigate to the site of interest though a subjects' vasculature.

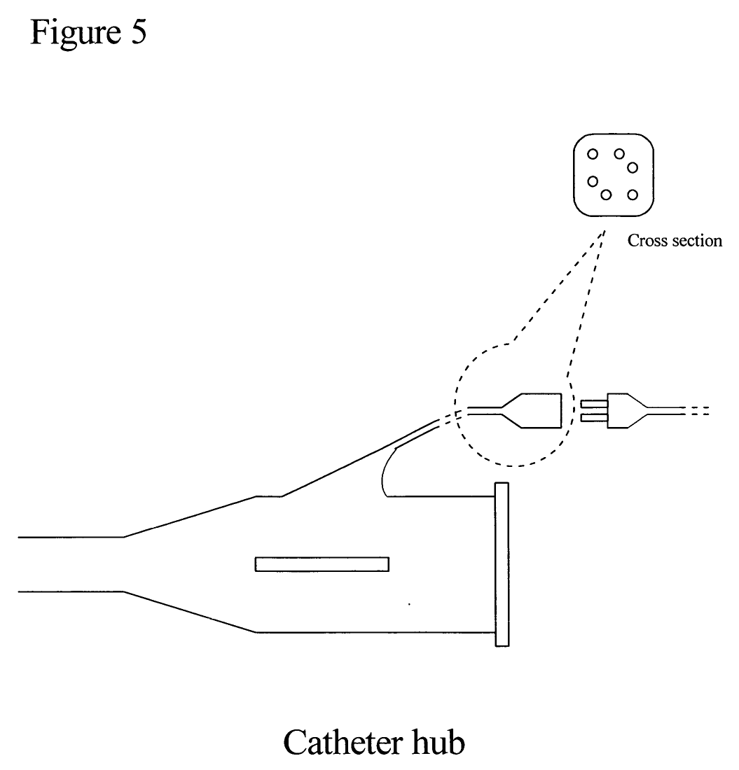

The conductive wires that provide current to heat the SMA wire in the steerable device generally travel along the shaft of the device and may connect to the steerable device to the driving device with a detachable connector, as shown in FIG. 5. The wires are preferably covered with an electric insulation coating. Where the steerable device is a catheter or another type of device with a lumen, the wires may exit the side of the catheter hub and terminate at the catheter hub with the connector. The connector may include a code or other means for identifying or registering the particular type of catheter with the driving device.

The form and shape of the driving device may vary as well. The driving device generally produces a driving signal and/or energy to heat the SMA wire as described herein. It is understood that a variety of types and forms of energy may be applied to heat the SMA material, including laser, electrical, mechanical, pneumatic, hydraulic, etc., or a combination thereof. In one embodiment, the driving device provides electrical energy to heat the SMA material incorporated into the steerable device. The electrical energy may be either DC or AC. In one embodiment, the driving device provides DC electrical energy to heat the SMA wire or wires. The DC electrical energy may be derived from an AC source, such as with circuitry adopted to convert incoming AC power into DC power, or directly from a battery, such as a rechargeable battery. The power may be derived from AC power and rechargeable battery combinations, in which instance the driving device may include circuitry to maintain a desired charge or battery level.

The driving device also includes circuitry that allows a user to control the steerable device in a graded manner as described herein. In one embodiment, the steerable device includes at least one or a plurality of SMA wires with each wire representing an individual channel that may be controlled independently. Thus, the driving device may include circuitry to allow a user to switch power supplied to each channel on and off, and to vary the amount of energy, e.g., the current, supplied to each of the channels. Alternatively, or in addition, the driving device or a computing device supplying a signal thereto may be programmed such that the driving device may supply current to a plurality of channels in one or more selectable sequences, which allows a user, for example, to selectively control the sequential generation of complicated shapes in the device.

The driving device may also be adopted to receive a data set, e.g., a three-dimensional data set, that represents an image or boundaries of the site of interest, e.g., the vascular tree or other types of body cavities, from imaging equipment, such an X-ray machine, computer tomography scanner, ultrasound, and magnetic resonance imaging unit, which may be updated in real-time during navigation. In one embodiment, the three-dimensional image of the site of interest is displayed to the user during navigation and the user or the device, or a combination thereof, can with the aid of the image register the catheter position in the three dimensional space and identify the target vessel. The driving device will, in one embodiment, then calculate the energy output to individual SMA wire or wires that is required to automatically bend the steerable device toward the identified target. The user interface may include switches, dials, joysticks, mouse pointers, alphanumeric keys, or any other interface means for switching and varying the energy to the channels or instructing the driving device to automatically bend the steerable device toward the target. The driving device may be connected to the steerable device with a universal or special purpose connector.

In one embodiment, the driving device includes circuitry and/or logic adopted to recognize the unique code of the steerable device. The driving device may then provide a particular driving signal and/or energy based on the type of steerable device connected to the driving device. For example, the device may be adopted to provide a signal to drive a plurality of different types of catheters with a different range of movement, number of SMA wires, compositions of SMA wire, resistance of SMA wire, lengths, shapes, etc. Thus, the driving device may be programmed to provide a driving signal to control a plurality of different types of catheters based on the characteristics of the particular steerable catheter. The driving device may also include an input/output interface to connect to other computers and/or to allow computer assisted catheterization and/or robotic control of the catheter.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A steerable device having a length, a proximal end, and a distal end, the steerable device comprising:
a structure composed of a material that exhibits shape memory characteristics incorporated into at least a portion of the length of the steerable device between the proximal end and the distal end, the structure having a length and a predetermined shape; and
at least one variable heating element disposed in at least a portion of the length of the structure to produce graded bending of the steerable device toward the predetermined shape by varying heat applied to the structure.

2. The device of claim 1, wherein the variable heating element comprises a heating coil having a variable coil density along at least a portion of the length of the device.

3. The device of claim 2, wherein the heating coil has a variable density that increases at least one of linearly, non-linearly, and in steps along at least a portion of the length of the device.

4. The device of claim 2, wherein the heating coil has a variable density that decreases from the distal end to the proximal end of the device to provide movement beginning with the distal end and continuing to the proximal end.

5. The device of claim 2, wherein the heating coil has a variable density that increases from the distal end to the proximal end of the device to provide movement beginning with the proximal end and continuing to the distal end.

6. The device of claim 2, wherein the heating coil comprises a first density and at least one other density different from the first density to provide movement in at least two stages.

7. The device of claim 2, wherein the heating coil comprises a first density, a second density, and at least one other density each different than the other to provide movement in at least three stages.

8. The device of claim 1, wherein the variable heating element comprises a joule heating element having a variable resistance along at least a portion of the length of the device.

9. The device of claim 8, wherein the structure serves as the joule-heating element.

10. The device of claim 9, wherein the structure comprises a first cross sectional area and at least one other cross sectional area different from the first thereby providing the variable resistance based on a variable cross sectional area.

11. The device of claim 9, wherein the structure is composed of a first composition having a first resistivity and at least one other composition having a resistivity that differs from the first thereby providing the variable resistance based on a variable composition.

12. The device of claim 1, wherein the variable heating element comprises a joule heating element, the structure serves as the joule heating element, and the structure is composed of a first composition having a first transformation temperature and at least one other composition having a transformation temperature that differs from the first.

13. A steerable device having a length, a proximal end, and a distal end, the steerable device comprising:
a plurality of structures composed of a material that exhibits shape memory characteristics incorporated into at least a portion of the length of the steerable device between the proximal end and the distal end, each of the structures having a length and a predetermined shape; and
a plurality of variable heating elements disposed in at least a portion of the length of the structure to produce graded bending of the steerable device toward at least one of the predetermined shapes by varying heat applied to at least one of the structures.

14. The device of claim 13, wherein the plurality of variable heating elements are joule heating elements, and wherein the plurality of structures serve as the joule heating elements.

15. The device of claim 13, wherein each of a plurality of the structures has a different predetermined shape.

16. The device of claim 13, wherein each of a plurality of the structures is disposed in different segments along the length of the device.

17. A system comprising:
a steerable device having a length, a proximal end, and a distal end, the steerable device comprising:
a plurality of structures composed of a material that exhibits shape memory characteristics incorporated into at least a portion of the length of the steerable device between the proximal end and the distal end, each of the structures having a length and a predetermined shape, and
a plurality of variable heating elements disposed in at least a portion of the length of the structure to produce graded bending of the steerable device toward at least one of the predetermined shapes by varying heat applied to at least one of the structures; and
a driving device that provides a drive signal to heat the plurality of the variable heating elements.

18. The system of claim 17, wherein the driving device provides a drive signal to heat a plurality of the variable heating elements at least one of individually and in combination.

19. The system of claim 17, wherein the driving device provides a drive signal to heat a plurality of the variable heating elements sequentially.

20. The system of claim 17, wherein the steerable device comprises at least three structures, and the predetermined shapes of structures allow a user to generate a vector force in any direction of a 360° space.

21. The system of claim 20, wherein at least one of the structures is disposed in the device for the predetermined shape of the structure to cause the structure, when heated, to generate a vector force perpendicular to a plane defined by at least two of the other structures.

22. The system of claim 20, wherein each of the three structures is disposed in the device for the predetermined shape of the structure to cause the structure, when heated, to generate a vector force perpendicular to a plane defined by at least two of the other structures.

23. The system of claim 17, wherein the driving device receives a data set representing boundaries of a site of interest and provides a drive signal to heat the plurality of the variable heating elements based at least in part of the data set.

24. The system of claim 17, wherein the steerable device comprises means for identifying the steerable device and wherein the driving device includes means for recognizing the steerable device and for producing a drive signal based on a type of steerable device connected to the driving device.
